(19) United States
(12) Patent Application Publication
(10) Pub. No.: US 2018/0017565 A1
(43) Pub. Date: Jan. 18, 2018

(54) PLASMA BIOMARKER FOR OVARIAN CANCER

(71) Applicant: ADELAIDE RESEARCH & INNOVATION PTY LTD, Adelaide (AU)

(72) Inventors: Martin K. Oehler, St Georges (AU); Carmela Ricciardelli, Parkside (AU); Noor Lokman, Forrestville (AU); Peter Hoffmann, Largs North (AU)

(21) Appl. No.: 15/546,223

(22) PCT Filed: Jan. 27, 2016
(86) PCT No.: PCT/AU2016/050040

§ 371 Date: Jul. 25, 2017

(30) Foreign Application Priority Data
Jan. 27, 2015 (AU) 2015900219

(51) Int. Cl. G01N 33/574

(52) U.S. Cl. CPC G01N 33/57449; G01N 2333/4718; G01N 2800/50

(57) ABSTRACT

The present invention relates to a biomarker for early stage ovarian cancer. Specifically, expression of the biomarker Annexin A2 is higher in the plasma of subjects with early stage ovarian cancer. Accordingly, methods of detecting early stage ovarian cancer in a subject, of identifying a subject having early stage ovarian cancer, and of determining if a subject is susceptible to developing ovarian cancer, are provided. Also provided are methods of screening candidate therapeutic agents for use in treating early stage ovarian cancer, and compositions and kits for detecting early stage ovarian cancer in a subject, for identifying a subject having early stage ovarian cancer, and for determining if a subject is susceptible to developing ovarian cancer.

### PRIORITY CLAIM

This application claims priority from Australian provisional patent application number 2015900219 filed on 27 Jan. 2015, the contents of which are to be taken as incorporated herein by this reference

### FIELD OF THE INVENTION

The present invention relates to the identification of a biological marker of early stage ovarian cancer. Specifically, an association between early stage ovarian cancer and an increased expression of Annexin A2 in plasma has been identified. Accordingly, Annexin A2 is a biological marker that can be utilised for a range of purposes including methods for detecting early stage ovarian cancer in a subject, methods for identifying a subject having early stage ovarian cancer, and methods for determining if a subject is susceptible to developing ovarian cancer.

### BACKGROUND OF THE INVENTION

Epithelial ovarian cancer is the leading cause of death from gynaecological malignancies and each year it is responsible for around 120,000 deaths worldwide. Indeed, it is the fifth leading cause of all cancer-related deaths in women. For example, it has been estimated that in the United States one woman in 72 will develop ovarian cancer in her lifetime, and that one woman in 96 will die of the disease. However, despite advances in surgery and chemotherapies, no substantial improvement in ovarian cancer survival has been observed over the last two decades.

The most widely recognised risk factors for ovarian cancer are menstrual, reproductive and hormonal factors. However, a number of other factors have also been linked to the development of ovarian cancer and these include diet, adult height, weight, and smoking. Furthermore, there have been clinical observations suggesting a genetic component to ovarian cancer risk due to familial aggregations of ovarian cancer. Indeed, women carrying BRCA1 and BRCA2 mutations have been seen to be at higher risk of developing ovarian cancer.

The high mortality rate of ovarian cancer arises due to the asymptomatic progression of the disease resulting in over 70% of cases being diagnosed in advanced stages (International Federation of Gynecology and Obstetrics (FIGO) stage III and IV) when the cancer has spread to the abdominal cavity or to other organs. Detection of ovarian cancer when it is still confined to the ovary (FIGO stage I) is associated with a 5-year survival rate of about 90% compared to less than 30% for women presenting with advanced ovarian disease (FIGO stage III/IV). Therefore, the detection of ovarian cancer at an early stage is the best way to improve overall survival from the disease. However, at present there are no clinically applicable tests and biological markers available for the early detection and screening of ovarian cancer.

To date, cancer antigen 125 (CA125) and HE4 are the only two protein-based biomarkers that have been clinically approved to distinguish benign from malignant ovarian lesions, to measure disease burden, and to evaluate ovarian cancer treatment. However, these markers are not elevated in all patients with ovarian cancer and may be increased in healthy women or women with benign diseases. Consequently, they do not have sufficient sensitivity and specificity for population-based risk assessment or early detection. For example, serum levels of CA125 greater than 35 U/mL in women presenting with an adnexal mass is the current gold standard for cancer diagnosis. However, elevated levels of CA125 are only observed in less than 50% of early stage patients and 70% of advanced stage patients. Consequently, CA125 is not a useful tumour marker alone for screening and early detection of ovarian cancer. The main utility of CA125 is for the differential diagnosis of ovarian masses and in cancer follow up.

There are several profound obstacles associated with traditional biomarker discovery focused on tumour-associated antigens. For example, there are quantitative obstacles in early-stage disease when the tumour is very small and therefore only very small quantities of the target antigen are produced (which might remain undetectable with currently available technology). There are also qualitative issues as the markers in many cases are incidental to the disease process and are often masked by the complexity of the examined biospecimens. The problem is further compounded by the genetic diversity of human populations and the influence of uncontrollable environmental factors meaning that potential biomarkers can be overshadowed by the high degree of natural variation in biomarker expression. Finally, obtaining a significant number of human samples of early-stage ovarian cancer for research is difficult due to the rarity of the disease diagnosed at this stage.

Given the lack of early detection tests and limitations of current approaches, there is an urgent need to identify new biomarkers for early stage ovarian cancer.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

The present invention is predicated, in part, on the determination that Annexin A2 is differentially expressed in the plasma of subjects with ovarian cancer. In particular, the inventors have found that expression of Annexin A2 is higher in plasma of subjects with early stage ovarian cancer compared to expression of Annexin A2 in plasma of normal subjects and in plasma of subjects in advanced stages of the disease. Annexin A2 is therefore a new plasma biomarker indicator of early stage ovarian cancer.

Accordingly, in a first aspect, the present invention provides a method of detecting early stage ovarian cancer in a subject, the method including:

(a) assessing an expression level of Annexin A2 in plasma of the subject;

(b) comparing the expression level of Annexin A2 in the plasma with a reference expression level for Annexin A2; and

(c) detecting early stage ovarian cancer in the subject on the basis of the comparison,

wherein an expression level of Annexin A2 in the subject that is higher than the reference expression level for Annexin A2 is indicative of early stage ovarian cancer in the subject.

In a second aspect, the present invention provides a method of identifying a subject having early stage ovarian cancer, the method including:

(a) assessing an expression level of Annexin A2 in plasma of the subject;

(b) comparing the expression level of Annexin A2 in the plasma with a reference expression level for Annexin A2; and

(c) identifying the subject as a subject having early stage ovarian cancer on the basis of the comparison,

wherein an expression level of Annexin A2 in the subject that is higher than the reference expression level for Annexin A2 indicatives that the subject has early stage ovarian cancer.

In a third aspect, the present invention provides a method of determining if a subject is susceptible to developing ovarian cancer, the method including:

(a) assessing an expression level of Annexin A2 in plasma of the subject;

(b) comparing the expression level of Annexin A2 in the plasma with a reference expression level for Annexin A2; and

(c) determining if the subject is susceptible to developing ovarian cancer on the basis of the comparison,

wherein an expression level of Annexin A2 in the subject that is higher than the reference expression level for Annexin A2 indicates that the subject is susceptible to developing ovarian cancer.

In some embodiments of the aforementioned aspects of the invention, the method is conducted in vitro.

In some embodiments of the aforementioned aspects of the invention, the expression level of Annexin A2 is assessed in a plasma sample obtained from the subject. In some embodiments, the expression level of Annexin A2 protein is assessed.

In some embodiments of the aforementioned aspects of the invention, the expression level of Annexin A2 protein is assessed using an antibody specific for the Annexin A2 protein.

In some embodiments of the first and second aspects of the invention, the expression level of tumour associated antigen CA125 is also assessed in serum or plasma of the subject. In one embodiment, the expression level of tumour associated antigen CA125 is assessed in a serum or plasma sample obtained from the subject.

In a fourth aspect, the present invention provides a method of screening a candidate therapeutic agent for use in treating early stage ovarian cancer in a subject, the method including the step of assaying the candidate therapeutic agent for activity in reducing the expression level of Annexin A2 in plasma of the subject.

In some embodiments of the fourth aspect of the invention, the method includes:

(a) administering the candidate therapeutic agent to the subject;

(b) assessing the expression level of Annexin A2 in plasma of the subject; and

(c) comparing the expression level of Annexin A2 in the plasma of the subject with the expression level of Annexin A2 in plasma of an untreated subject having early stage ovarian cancer,

wherein if the expression level of Annexin A2 in the subject is lower than the expression level of Annexin A2 in the untreated subject, the candidate therapeutic agent is useful for treating early stage ovarian cancer.

In some embodiments of the fourth aspect of the invention, the method includes:

(a) assessing the expression level of Annexin A2 in plasma of a subject who has been administered the candidate therapeutic agent; and

(b) comparing the expression level of Annexin A2 in the plasma of the subject with the expression level of Annexin A2 in plasma of an untreated subject having early stage ovarian cancer,

wherein if the expression level of Annexin A2 in the subject is lower than the expression level of Annexin A2 in the untreated subject, the candidate therapeutic agent is useful for treating early stage ovarian cancer.

In some embodiments of the fourth aspect of the invention, the expression level of Annexin A2 is assessed in a plasma sample obtained from the subject.

In a fifth aspect, the present invention provides a method of screening a candidate therapeutic agent for use in treating early stage ovarian cancer in a subject, the method including:

(a) exposing the candidate therapeutic agent to a cell expressing Annexin A2;

(b) measuring for a change in the expression level of Annexin A2 in the cell; and

(c) comparing the expression level of Annexin A2 in the cell to a reference expression level for Annexin A2,

wherein if the expression level of Annexin A2 in the cell is lower than the reference expression level for Annexin A2, the candidate therapeutic agent is useful for treating early stage ovarian cancer in a subject.

In some embodiments of the fourth or fifth aspects of the invention, the expression level of Annexin A2 protein is assessed. In some embodiments, the expression level of Annexin A2 protein is assessed using an antibody specific for the Annexin A2 protein.

In a sixth aspect, the present invention provides a composition for detecting early stage ovarian cancer in a subject, for identifying a subject having early stage ovarian cancer, or for determining if a subject is susceptible to developing ovarian cancer, the composition including an agent that binds to, or interacts with, Annexin A2 present in plasma of the subject.

In some embodiments of the sixth aspect of the invention, the agent is an antibody specific for the Annexin A2 protein. In some embodiments, the composition further includes an agent that binds to, or interacts with, tumour associated antigen CA125.

In a seventh aspect, the present invention provides a kit for detecting early stage ovarian cancer in a subject, for identifying a subject having early stage ovarian cancer, or for determining if a subject is susceptible to developing ovarian cancer, the kit including means for assessing the expression level of Annexin A2 in plasma of the subject.

In some embodiments of the seventh aspect of the invention, the kit includes an agent that binds to, or interacts with, Annexin A2 present in plasma of the subject. In some embodiments, the agent is an antibody specific for the Annexin A2 protein. In some embodiments, the kit further includes means for assessing the expression level of tumour associated antigen CA125 in serum or plasma of the subject.

In an eighth aspect, the present invention provides a biomarker of early stage ovarian cancer, the biomarker being Annexin A2. In one embodiment, an expression level of Annexin A2 in a subject that is higher than a reference expression level for Annexin A2 is indicative of early stage ovarian cancer in the subject.

### BRIEF DESCRIPTION OF THE FIGURES

For a further understanding of the aspects and advantages of the present invention, reference should be made to the following detailed description, taken in conjunction with the accompanying figures.

FIG. 1—is a graph comparing Annexin A2 expression levels in matching serum and plasma samples (n=23).

FIG. 2—is a graph comparing Annexin A2 expression levels in serum from subjects with normal ovaries, benign ovarian tumours, early stage ovarian carcinomas, and advanced stage ovarian carcinomas.

FIG. 3—is a graph comparing Annexin A2 expression levels in plasma from subjects with normal ovaries, benign ovarian tumours, early stage ovarian carcinomas, and advanced stage ovarian carcinomas. * P<0.05, significantly increased compared to other patient groups (Kruskal Wallis test, Dunns Multiple comparison test).

FIG. 4—is a receiver-operating characteristic (ROC) curve and area under the curve of plasma Annexin A2 for discriminating early stage ovarian cancers (n=11) from controls (normal ovaries and benign ovarian tumours, n=36) (AUC=0.995, 95% CI 0.98-1.00, P<0.0001).

### DETAILED DESCRIPTION OF THE INVENTION

The present invention is predicated, in part, on the identification of a protein (Annexin A2), the expression level of which is altered in plasma of subjects with early stage ovarian cancer. The differential expression of this protein indicates that it is a suitable biomarker which can form the basis of tests for detecting early stage ovarian cancer in subjects.

It is to be made clear that certain disclosed embodiments of the present invention have one or more combinations of advantages. For example, some of the advantages of the embodiments disclosed herein include one or more of the following: a sensitive and specific biomarker for detecting early stage ovarian cancer; an improved biomarker for ovarian cancer; an improved method for detecting early stage ovarian cancer; an improved method for identifying a subject having early stage ovarian cancer; a method for determining if a subject is susceptible to developing ovarian cancer; methods for screening a candidate therapeutic agent for use in treating early stage ovarian cancer; improved compositions and kits for detecting early stage ovarian cancer; to address one or more problems in the art; to provide one or more advantages in the art; and/or to provide a useful commercial choice. Other advantages of certain embodiments are disclosed herein.

A biomarker is effectively an organic biomolecule which is differentially present in a sample taken from a subject of one phenotypic status (e.g. having a disease) as compared with another phenotypic status (e.g. not having the disease). A biomarker is differentially present between different phenotypic status groups if the mean or median expression level or amount of the biomarker is calculated to be different (e.g. higher or lower) between the groups. Therefore, biomarkers, alone or in combination, provide an indication that a subject belongs to one phenotypic status or another. With respect to biomarkers that are specific for a particular type of cancer, generally these would only be expected to be present in a subject having that cancer, but not present in a “normal” subject not having that cancer. However, it is to be made clear that the biomarker of the present invention (Annexin A2) is differentially expressed at the earliest stages of ovarian cancer development and therefore is present in higher amounts in affected subjects who considered themselves “normal” due to lack of phenotypic expression of the ovarian cancer.

Accordingly, in a first aspect, the present invention provides a method of detecting early stage ovarian cancer in a subject, the method including:

(a) assessing an expression level of Annexin A2 in plasma of the subject;

(b) comparing the expression level of Annexin A2 in the plasma with a reference expression level for Annexin A2; and

(c) detecting early stage ovarian cancer in the subject on the basis of the comparison,

wherein an expression level of Annexin A2 in the subject that is higher than the reference expression level for Annexin A2 is indicative of early stage ovarian cancer in the subject.

In a second aspect, the present invention provides a method of identifying a subject having early stage ovarian cancer, the method including:

(a) assessing an expression level of Annexin A2 in plasma of the subject;

(b) comparing the expression level of Annexin A2 in the plasma with a reference expression level for Annexin A2; and

(c) identifying the subject as a subject having early stage ovarian cancer on the basis of the comparison,

wherein an expression level of Annexin A2 in the subject that is higher than the reference expression level for Annexin A2 indicatives that the subject has early stage ovarian cancer.

As indicated above, the present inventors have determined that Annexin A2 is differentially expressed at an early stage in the development of ovarian cancer. Accordingly, in a third aspect, the present invention provides a method of determining if a subject is susceptible to developing ovarian cancer, the method including:

(a) assessing an expression level of Annexin A2 in plasma of the subject;

(b) comparing the expression level of Annexin A2 in the plasma with a reference expression level for Annexin A2; and

(c) determining if the subject is susceptible to developing ovarian cancer on the basis of the comparison,

wherein an expression level of Annexin A2 in the subject that is higher than the reference expression level for Annexin A2 indicates that the subject is susceptible to developing ovarian cancer.

In one embodiment of the aforementioned aspects of the present invention, the early stage ovarian cancer is stage I or stage II carcinoma. The stage of a cancer is determined by the extent to which the cancer has spread. Stage I ovarian cancer is defined by the cancerous cells being localised to the ovary with the cancerous cells yet to spread to the pelvic surfaces or pelvic organs (stage II), abdominal cavity or lymph nodes (stage III), and distant organs (stage IV). Accordingly, stage I ovarian cancer is localised to one or both ovaries.

The meaning of “ovarian cancer” would be well understood by a person skilled in the art. For the avoidance of doubt, an ovarian cancer is a cancerous growth arising from the ovary. More than 90% of ovarian cancers are epithelial in origin given that they originate from the surface of the ovary. However, it is believed that the fallopian tubes and the peritoneum may also be the source of some ovarian cancers. Ovarian cancers are also categorised as gynaecological cancers.

As used herein “Annexin A2” refers to a member of the annexin family, a group of calcium-dependent phospholipid-binding proteins that play a role in the regulation of cellular growth and in signal transduction pathways. The Annexin A2 gene has been found in a number of species, including human, mouse, rat, cow, chicken, dog, pig, Rhesus monkey, sheep, chimpanzee, camel, horse, zebrafish, frog and shark. Annexin A2 is also referred to in the art as Annexin-2, protein I, Annexin II, lipocortin II, chromobindin 8, chromobindin-8, calpactin I heavy chain, calpactin-1 heavy chain, calpactin I heavy polypeptide, placental anticoagulant protein IV, epididymis secretory protein Li 270, P36, ANXA2, ANX2, LIP2, LPC2, CAL1H, LPC2D, ANX2L4, PAP-IV and HEL-S-270.

Details regarding Annexin A2 may be accessed from the GenBank database at the National Center for Biotechnology Information (NCBI) (www.ncbi.nlm.nih.gov). For example, the Gene ID number for human Annexin A2 is 302. The human Annexin A2 gene encodes four isoform variants represented by GenBank Accession Numbers NM_001002858.2 and NP_001002858.1 (variant 1), NM_001002857.1 and NP_001002857.1 (variant 2), NM_004039.2 and NP_004030.1 (variant 3), and NM_001136015.2 and NP_001129487.1 (variant 4). Further details of the Annexin A2 gene in other species may be accessed from the NCBI. For example, the Gene ID number for chimpanzee Annexin A2 is 747018, for Rhesus monkey is 706240, for mouse is 12306, for dog is 403435, for horse is 100054320, and for cow is 282689.

Further details regarding the Annexin A2 gene in humans and other species can also be found at the UniGene portal of the NCBI (i.e. UniGene Hs. 511605—http://www.ncbi.nlm.nih.gov/UniGene/clust.cgi?ORG=Hs&CID=511605). Alternatively, details of the nucleotide and amino acid sequence for Annexin A2 can be accessed from the UniProt database (www.uniprot.org) wherein the UniProt ID for human Annexin A2 is P07355 (variant 1—http://www.uniprot.org/uniprot/P07355), and A0A024R5Z7 (variants 2 to 4—http://www.uniprot.org/uniprot/A0A024R5Z7).

It is to be made clear that reference herein to Annexin A2 includes a reference to its naturally-occurring variants. In this regard, a “variant” of Annexin A2 may exhibit a nucleic acid or an amino acid sequence that is at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, or at least about 99.9% identical to native Annexin A2. In some embodiments, a variant of Annexin A2 is expected to retain native biological activity or a substantial equivalent thereof.

The methods of the aspects of the invention referred to above require assessing (or measuring) an expression level of Annexin A2 in a subject. As would be understood by a person skilled in the art, Annexin A2 is a functional biomolecule composed primarily of amino acids, with the amino acid sequence determined by a corresponding Annexin A2 gene. Accordingly, as used herein, the term “assessing an expression level” of Annexin A2 includes: (1) measuring the level of transcription of the corresponding Annexin A2 gene into a messenger RNA (mRNA) molecule; and/or (2) measuring the level of translation of the mRNA into the protein (i.e. measuring the level of protein per se); and/or (3) measuring the level of activity of the translated protein. In effect, the expression level of Annexin A2 can be measured at the RNA and/or protein stages of expression. In one embodiment the “expression level” of Annexin A2 protein in the subject may be a reflection of the extent of translation of the Annexin A2 gene in the subject.

Accordingly, the term “biomarker” as used herein includes, but is not limited to, Annexin A2 proteins (polypeptides), Annexin A2 polynucleotides (e.g. mRNA) and/or Annexin A2 metabolites whose expression level (e.g. level of transcription, level of translation, and/or level of activity) in a sample from a subject with early stage ovarian cancer is higher than the expression level of the same biomarker in a normal sample or late stage ovarian cancer sample (i.e. a reference expression level).

The term “gene” is to be understood to mean a region of genomic nucleotide sequence associated with a coding region which is transcribed and translated into the protein. Accordingly, the term “gene” with respect to Annexin A2 may include regulatory regions (e.g. promoter regions), transcribed regions, protein coding exons, introns, untranslated regions and other functional and/or non-functional sequence regions associated with Annexin A2.

Measuring the level of Annexin A2 protein (i.e. measuring the level of translation of Annexin A2 mRNA into protein) in a subject can be achieved a number of ways as would be understood by a person skilled in the art. Exemplary methods include, but are not limited to, antibody-based (immunoassay-based) testing techniques (including Western blotting, immunoblotting, enzyme-linked immunosorbant assay (ELISA), “sandwich” immunoassays, radioimmunoassay (RIA), immunoprecipitation and dissociation-enhanced lanthanide fluoro immuno assay (DELFIA), precipitin reactions, gel diffusion precipitin reactions, immunodiffusion assays, immunoradiometric assays and protein A immunoassays), polystyrene and/or bead-based assays (such as Singleplex and Miltiplex Luminex® assays), protein microarrays, mass spectrometry-based techniques (including liquid chromatography coupled to tandem mass spectrometry (LC-MS/MS), nano LC-MS/MS, matrix-assisted laser desorption/ionization mass spectrometry (MALDI-MS) as described in WO 2009/004576 (including surface enhanced laser desorption/ionization mass spectrometry (SELDI-MS), especially surface-enhanced affinity capture (SEAC), surface-enhanced need desorption (SEND) or surface-enhanced photo label attachment and release (SEPAR)), matrix-assisted laser desorption/ionization time-of-flight (MALDI-TOF) mass spectrometry, proteomics techniques, surface plasmon resonance (SPR), versatile fibre-based SPR, chemiluminescence, fluorescent polarization, phosphorescence, immunohistochemistry, immunofluorescence, microcytometry, microscopy, fluorescence activated cell sorting (FACS), and flow cytometry. Some of these techniques are described in further detail below.

With respect to antibody-based testing methods such as immunohistochemistry and immunoblotting, antibodies or antisera specific for Annexin A2 can be used to detect protein levels. These techniques typically rely on the antibodies being detectably labelled. The antibody can be labelled by covalently or non-covalently combining the antibody with a substance or ligand that provides, or enables the generation of, a detectable signal. Some examples include, but are not limited to, radioactive isotopes, enzymes, fluorescent substances, luminescent substances, ligands, microparticles, redox molecules, substrates, cofactors, inhibitors, magnetic particles and the like. Examples of the radioactive isotopes include, but are not limited to, ³H, ¹²C, ¹³C, ³²P, ³⁵S, ³⁶Cl, ⁵¹Cr, ⁶⁷Co, ⁵⁸Co, ⁵⁹Fe, ⁹⁰Y, ¹²⁵I, ¹³¹I, and ¹⁸⁶Re. Examples of enzymes available as detection labels include, but are not limited to, β-glucuronidase, β-glucosidase, β-galactosidase, urease, peroxidase or alkaline phosphatase, acetylcholinesterase, glucose oxidase, hexokinase and GDPase, RNase, glucose oxidase and luciferase, phosphofructokinase, phosphoenolpyruvate carboxylase, aspartate aminotransferase, phosphenolpyruvate decarboxylase, and β-latamase. Examples of the fluorescent substances include, but are not limited to, fluorescin, isothiocyanate, rhodamine, phycoerythrin, phycocyanin, allophycocyanin, o-phthaldehyde and fluorescamin. Examples of luminescent substances include, but are not limited to, acridinium esters, luciferin and luciferase. Examples of ligands include, but are not limited to, biotin and its derivatives. Examples of the microparticles include, but are not limited to, colloidal gold and colored latex. Examples of the redox molecules include, but are not limited to, ferrocene, ruthenium complexes, viologen, quinone, Ti ions, Cs ions, diimide, 1,4-benzoquinone, hydroquinone, K₄W(CN)₈, [Os(bpy)₃]²⁺, [RU(bpy)₃]²⁺, and [MO(CN)₈]⁴⁻. Alternatively, unlabelled primary antibody may be used in conjunction with a labelled secondary antibody that is specific for the primary antibody. Immunohistochemistry protocols and kits are well known in the art and are commercially available.

Also contemplated are traditional immunoassays including, for example, sandwich immunoassays including ELISA or fluorescence-based immunoassays, as well as other enzyme immunoassays. Nephelometry is an assay performed in liquid phase, in which Annexin A2 antibodies are in solution. Binding of Annexin A2 protein to the antibody results in changes in absorbance, which are measured. In the SELDI-based immunoassay, a biospecific capture reagent for Annexin A2 is attached to the surface of an MS probe, such as a pre-activated ProteinChip array (see below). The protein is then specifically captured on the biochip through this reagent, and the captured protein is detected by mass spectrometry (see below).

The term “antibody” is used herein in the broadest sense and encompasses intact polyclonal antibodies, intact monoclonal antibodies, antibody fragments (such as linear antibodies, single-chain antibody molecules, Fc or Fc′ peptides, Fab, Fab′, F(ab′)2, and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be one of any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations.

Naturally occurring immunoglobulins have a common core structure in which two identical light chains (about 24 kD) and two identical heavy chains (about 55 or 70 kD) form a tetramer. The amino-terminal portion of each chain is known as the variable (V) region and can be distinguished from the more conserved constant (C) regions of the remainder of each chain. Within the variable region of the light chain is a C-terminal portion known as the J region. Within the variable region of the heavy chain, there is a D region in addition to the J region. Most of the amino acid sequence variation in immunoglobulins is confined to three separate locations in the V regions known as hypervariable regions or complementarity determining regions (CDRs) which are directly involved in antigen binding. Proceeding from the amino-terminus, these regions are designated CDR1, CDR2 and CDR3, respectively. The CDRs are held in place by more conserved framework regions (FRs). Proceeding from the amino-terminus, these regions are designated FR1, FR2, FR3, and FR4, respectively. The locations of CDR and FR regions and a numbering system have been defined for example by Kabat et al., 1991 (Sequences of Proteins of Immunological Interest, Fifth Edition, U.S. Department of Health and Human Services, U.S. Government Printing Office).

The antigen binding part of the antibody is to be understood to mean the antigen-binding portion of the antibody molecule, including a Fab, Fab′, F(ab′)₂, Fv, a single-chain antibody (scFv), a chimeric antibody, a diabody or any polypeptide that contains at least a portion of an immunoglobulin that is sufficient to confer specific antigen binding, such as a molecule including one or more CDRs (see further detail below).

Antibodies exist as intact immunoglobulins or as a number of well-characterized fragments produced by digestion with various peptidases. Therefore, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, a person skilled in the art would appreciate that such fragments may be synthesized de novo either chemically or by using recombinant DNA methodology. Therefore, the term antibody, as used herein, also includes antibody fragments either produced by the modification of whole antibodies, or those synthesized de novo using recombinant DNA methodologies (e.g. single chain Fv) or those identified using phage display libraries (see for example McCafferty et al., 1990, Nature 348: 552-554).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g. an enzyme, toxin, hormone, growth factor, drug, etc.; or (b) the variable region, or a portion thereof, is altered, replaced or exchanged with a variable region having a different or altered antigen specificity. The chimeric antibodies may be monovalent, divalent, or polyvalent immunoglobulins. For example, a monovalent chimeric antibody is a dimer (HL) formed by a chimeric H chain associated through disulfide bridges with a chimeric L chain, as noted above. A divalent chimeric antibody is a tetramer (H₂ L₂) formed by two HL dimers associated through at least one disulfide bridge. A polyvalent chimeric antibody is based on an aggregation of chains.

In some embodiments, the antibody may be a humanised antibody. A “humanised” antibody is an antibody that retains the reactivity of a non-human antibody while being less immunogenic in humans. This can be achieved, for example, by retaining the non-human CDR regions and replacing the remaining parts of the antibody with their human counterparts. See for example Morrison et al., 1984, Proc. Natl. Acad. Sci. USA, 81: 6851-6855; Morrison and Oi, 1988, Adv. Immunol., 44: 65-92; Verhoeyen et al., 1988, Science, 239: 1534-1536; Padlan, 1991, Molec. Immun., 28: 489-498; and Padlan, 1994, Molec. Immun., 31: 169-217.

In some embodiments, the antibody may be a fully human antibody. As would be understood by a person skilled in the art, a fully human antibody is an antibody in which both the variable and constant regions are of human origin. Methods for producing or identifying such antibodies are described below.

Additional antibody types are also contemplated by the present invention. These include antibodies sourced from a non-mammalian animal such as a cartilaginous fish (e.g. shark IgNAR antibodies—see WO2012/073048) or modified human protein scaffolds that provide functionality similar to shark antibodies, such as i-bodies which have binding moieties based on shark IgNAR antibodies (see WO2005/118629). IgNARs are disulphide-bonded homodimers consisting of five constant domains (CNAR), one variable domain (VNAR), and no light chains (Greenberg et al., 1995, Nature 374: 168-173; Nuttall et al., 2001, Mol. Immunol., 38: 313-326; Diaz et al., 2002, Immunogenetics 54: 501-512; and Nuttall et al., 2003, Eur. J. Biochem., 270: 3543-3554). Antibodies sourced from camels (camelid antibodies), dromedaries and llamas are also contemplated by the present invention. Such antibodies consist of only two heavy chains and are devoid of light chains. Due to the heavy chain dimer structure of camelid and shark antibodies, they are sometimes termed “heavy-chain mini-antibodies” (mnHCAbs) or “mini-antibodies” (mnAbs) (Holliger and Hudson, 2005, Nat. Biotechnol., 23(1): 1126-1136). Without the light chain, these antibodies bind to their antigens by a single domain—the variable antigen binding domain of the heavy chain immunoglobulin, referred to as Vab (camelid antibodies) or VNAR (shark antibodies).

Affibodies are also contemplated by the present invention. Affibody molecules are a class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord K et al., 1997, Nat. Biotechnol., 15: 772-777; Ronmark J et al., 2002, Eur. J. Biochem., 269: 2647-2655). Further details about Affibodies and methods of production thereof are also disclosed in U.S. Pat. No. 5,831,012.

Antibodies for any of the methods and applications referred to herein can be produced according to well-established techniques in the art. For example, various hosts including rabbits, rats, goats, mice, humans, and others may be immunised by injection with Annexin A2 polypeptide or with any fragment, peptide or oligopeptide thereof which has immunogenic properties. Various adjuvants may be used to increase immunological response and include, but are not limited to, Freund's, mineral gels such as aluminum hydroxide, and surface-active substances such as lysolecithin. Adjuvants used in humans include BCG (bacilli Calmette-Guerin) and Corynebacterium parvum.

It is preferred that the Annexin A2 oligopeptides, peptides, or fragments used to induce antibodies have an amino acid sequence consisting of at least 5 amino acids, and, more preferably, of at least 10 amino acids of Annexin A2. It is also preferable that these oligopeptides, peptides, or fragments are identical to a portion of the amino acid sequence of the natural protein and contain the entire amino acid sequence of a small, naturally occurring molecule. Short stretches of amino acids from Annexin A2 may be fused with those of another protein, such as keyhole limpet haemocyanin (KLH), and antibodies to the chimeric molecule may be produced.

Monoclonal antibodies to Annexin A2 may be prepared using any technique which provides for the production of antibody molecules by continuous cell lines in culture. These include, but are not limited to, the hybridoma technique, the human B-cell hybridoma technique, and the EBV-hybridoma technique (for example, see Kohler et al., 1975, Nature 256: 495-497; Kozbor et al., 1985, J. Immunol. Methods 81:31-42; Cote et al., 1983, Proc. Natl. Acad. Sci. USA 80: 2026-2030; and Cole et al., 1984, Mol. Cell Biochem. 62: 109-120).

Antibodies may also be produced by inducing in vivo production in the lymphocyte population or by screening immunoglobulin libraries or panels of highly specific binding reagents as disclosed in the literature (for example, see Orlandi et al., 1989, Proc. Natl. Acad. Sci. USA 86: 3833-3837; and Winter and Milstein, 1991, Nature 349: 293-299). Antibodies may also be generated using phage display. For example, functional antibody domains are displayed on the surface of phage particles that carry the polynucleotide sequences encoding them. Such phage can be utilized to display antigen-binding domains expressed from a repertoire or combinatorial antibody library (e.g. human or murine). Phage expressing an antigen binding domain that binds Annexin A2 can be selected or identified using the Annexin A2 protein or a portion thereof. Phage used in these methods are typically filamentous phage including fd and MI 3 binding domains expressed from phage with Fab, Fv or disulfide stabilised Fv antibody domains recombinantly fused to either the phage gene III or gene VIII protein. Examples of phage display methods that can be used to make Annexin A2 antibodies may include those disclosed in Brinkman et al., 1995, J. Immunol. Methods 182: 41-50; Ames et al., 1995, J. Immunol. Methods 184: 177-186; Kettleborough et al., 1994, Eur. J. Immunol. 24: 952-958; Persic et al., 1997, Gene 187: 9-18; Burton et al., 1994, Advances in Immunology 57: 191-280; PCT application number PCT/GB91/01134; PCT publications numbers WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

Techniques which can be used to produce single-chain Fvs and antibodies include those described in U.S. Pat. Nos. 4,946,778 and 5,258,498; Huston et al., 1991, Methods in Enzymology 203: 46-88; Shu et al., 1993, Proc. Natl. Acad. Sci. USA 90: 7995-7999; and Skerra et al., 1988, Science 240: 1038-1040.

Antibody fragments which contain specific binding sites for Annexin A2 may be generated using standard techniques known in the art. For example, F(ab′)2 fragments may be produced by pepsin digestion of an Annexin A2 antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (for example, see Huse et al., 1989, Science 246: 1275-1281).

Fully human Annexin A2 antibodies may be produced using a number of techniques. These include using display technologies as mentioned above in which human antibodies or antibody fragments are displayed on the surface of a phage for example. In another method (Lonberg N, 2008, Handb. Exp. Pharmacol., 69-97), first generation human antibodies to Annexin A2 may be produced by utilising transgenic animals that produce antibodies from human genes. When challenged with an antigen (i.e. Annexin A2 or an oligopeptide, peptide, or fragment thereof), these animals produce human antibodies avoiding the humanisation steps. Human antibodies can also be produced from B cells isolated from humans using a technique described in Crowe J E Jr, 2009, Vaccine 27: 47-51. Other techniques for human antibody production are described in PCT international publication number WO 2013/168150 and Duvall M et al., 2011, mAbs 3(2): 203-208, amongst others. For example, Duvall et al utilises technology which produces human IgG antibody libraries from naïve B cells isolated from human tonsil tissue. The antibodies are produced from human genes and are therefore 100% human antibodies.

A further technique for measuring the level of Annexin A2 protein using an antibody-based platform involves the versatile fibre-based surface plasmon resonance (VeSPR) biosensor, as described in PCT International Publication No. WO 2011/113085. Traditional SPR is a well-established method for label-free bio-sensing that relies on the excitation of free electrons at the interface between a dielectric substrate and a thin metal coating. The condition under which the incoming light couples into the plasmonic wave depends on the incidence angle and the wavelength of the incoming light as well as the physical properties (dielectric constant/refractive index) of the sensor itself and the surrounding environment. For this reason, SPR is sensitive to even small variations in the density (refractive index) in the close vicinity of the sensor, and does not require the use of fluorescent labels. The small variation of refractive index induced by the binding biomolecules such as proteins onto the sensor surface, can be measured by monitoring the coupling conditions via either the incidence angle or the wavelength of the incoming light. Existing SPR systems use the bulky and expensive Krestchmann prism configuration where one side of the prism is coated with a metal such as gold or silver that can support a plasmonic wave. Alternative SPR architectures have been developed based on optical fibres with the metallic coating deposited around a short section of the fibre. This approach reduces the complexity and cost of such sensors, opening a pathway to distinctive applications such as dip sensing. The material at the sensor surface is probed by monitoring the wavelength within a broad spectrum that is absorbed due to coupling to the surface plasmon. These techniques suffer from practical limitations associated with the need for careful temperature calibration, causing difficulty in analysing large numbers of samples consistently. VeSPR is a powerful variant of an optical-fibre based SPR sensor. VeSPR has a number of demonstrated advantages over existing SPR techniques including: (i) higher signal-to-noise ratio thus higher sensitivity; (ii) self-referencing of the transducing signal thus avoiding expensive/bulky temperature control; and (iii) the ability to perform multiplexed detection of different analytes using a single fibre.

Proteomics can also be used to measure the level of Annexin A2 protein in a sample at a certain point of time. In particular, proteomic techniques can be used to assess the global changes of protein expression in a sample (also referred to as expression proteomics). Proteomic analysis typically includes: (i) separation of individual proteins in a sample by 2-D gel electrophoresis (2-D PAGE); (ii) identification of the individual polypeptides recovered from the gel, for example by mass spectrometry or N-terminal sequencing; and (iii) analysis of the data using bioinformatics.

Protein microarrays (also termed biochips) may also be used to determine the level of Annexin A2 protein in a sample. Many protein biochips are described in the art, including for example protein biochips produced by Ciphergen Biosystems, Inc. (Fremont, Calif.), Zyomyx (Hayward, Calif.), Invitrogen (Carlsbad, Calif.), Biacore (Uppsala, Sweden) and Procognia (Berkshire, UK). Examples of such protein biochips are described in the following patents or published patent applications: U.S. Pat. Nos. 6,225,047, 6,537,749, 6,329,209, and 5,242,828, and PCT International Publication Nos. WO 00/56934 and WO 03/048768.

The level of Annexin A2 protein can also be measured by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these. The mass spectrometer may be a laser desorption/ionization mass spectrometer. In laser desorption/ionization mass spectrometry, a sample containing the Annexin A2 protein is placed on the surface of a mass spectrometry probe, a device adapted to engage a probe interface of the mass spectrometer and to present the protein to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of Annexin A2 protein by LDI can take the form of matrix-assisted laser desorption/ionization (MALDI—as described for example in Karas M and Hillenkamp F, 1988, Anal. Chem., 60: 2299-2301; Tanaka K et al., 1988, Rapid Commun. Mass Spectrom., 2: 151-153; and Norris J L and Caprioli R M, 2013, Chem Rev., 113: 2309-2342) or of surface-enhanced laser desorption/ionization (SELDI—as described for example in Hutchens T and Yip T, 1993, Rapid Commun. Mass Spectrom., 7: 576-580; Tang N et al., 2004, Mass Spec. Reviews, 23: 34-44; and U.S. Pat. Nos. 5,719,060 and 6,225,047).

Other laser desorption mass spectrometry methods which may be employed include surface-enhanced neat desorption (SEND—as described for example in U.S. Pat. No. 6,124,137 and PCT International Publication No. WO 03/64594), SEAC/SEND (a version of laser desorption mass spectrometry in which both a capture reagent and an energy absorbing molecule are attached to the sample presenting surface), and surface-enhanced photolabile attachment and release (SEPAR—which involves the use of probes having moieties attached to the surface that can covalently bind Annexin A2 protein, and then release the protein through breaking a photolabile bond in the moiety after exposure to light, e.g. to laser light).

With respect to measuring the level of activity of Annexin A2 protein, assays which exploit the known activity of Annexin A2 may be employed. For example, Annexin A2 is known to bind with tissue plasminogen activator on the cell surface to mediate the conversion of plasminogen to plasmin. Therefore, assays which measure the level of plasmin production by Annexin A2 in the presence of tissue plasminogen activator will be a reflection of the level and/or activity of Annexin A2. Annexin A2 has also been shown to interact with tenascin C and this interaction regulates cell migration and enhances cell proliferation of endothelial cells. Accordingly, assays which measure endothelial cell proliferation in the presence of Annexin A2 and tenascin C will be a reflection of the level and/or activity of Annexin A2 in a particular sample. Annexin A2 has also been shown to complex with S100A10 on the surface of cells potentially facilitating cell-cell interactions. Accordingly, methods which assay for cell-cell interactions in the presence of Annexin A2 and S100A10 will be a reflection of the level and/or activity of Annexin A2 in a particular sample. These are non-limiting examples and any other method which exploits the activity of Annexin A2 may be employed.

Methods for measuring the level of transcription of the Annexin A2 gene into mRNA are also known in the art. For example, levels of mRNA may be measured by techniques which include, but are not limited to, Northern blotting, RNA in situ hybridisation, reverse-transcriptase PCR (RT-PCR), real-time (quantitative) RT-PCR, microarrays, or “tag based” technologies such as SAGE (serial analysis of gene expression). Microarrays and SAGE may be used to simultaneously quantitate the expression of more than one gene. Primers or probes may be designed based on nucleotide sequences of the genes or transcripts thereof. Methodology similar to that disclosed in Paik et al., 2004 (NEJM, 351(27): 2817-2826), or Anderson et al., 2010 (Journal of Molecular Diagnostics, 12(5): 566-575) may be used to measure the expression of Annexin A2 and one or more other genes of interest. Many methods are also disclosed in standard molecular biology text books such as Green M R and Sambrook J, Molecular Cloning: A Laboratory Manual (4th edition), Cold Spring Harbor Laboratory Press, 2012.

With respect to RT-PCR, the first step is typically the isolation of total RNA from a sample obtained from the subject under investigation. A typical sample in this instance would be a tissue biopsy from an ovary or an ovarian tumour sample (and corresponding normal tissue if possible), although other sample sources are contemplated as described below. If the source of RNA is from a tumour, RNA can also be extracted, for example, from frozen or archived paraffin-embedded and fixed (e.g. formalin-fixed) tissue samples previously obtained from the subject. Messenger RNA (mRNA) may be subsequently purified from the total RNA sample. The total RNA sample (or purified mRNA) is then reverse transcribed into cDNA using a suitable reverse transcriptase. The reverse transcription step is typically primed using oligo-dT primers, random hexamers, or primers specific for the Annexin A2 gene, depending on the RNA template. The cDNA derived from the reverse transcription reaction then serves as a template for a typical PCR reaction. In this regard, two oligonucleotide PCR primers specific for the Annexin A2 gene are used to generate a PCR product. A third oligonucleotide, or probe, designed to detect a nucleotide sequence located between the other two PCR primers is also used in the PCR reaction. The probe is non-extendible by the Taq DNA polymerase enzyme used in the PCR reaction, and is labelled with a reporter fluorescent dye and a quencher fluorescent dye. Any laser-induced emission from the reporter dye is quenched by the quenching dye when the two dyes are located close together, as they are on the probe. During the PCR amplification reaction, the Taq DNA polymerase enzyme cleaves the probe in a template-dependent manner. The resultant probe fragments disassociate in solution, and signal from the released reporter dye is freed from the quenching effect of the second fluorophore. One molecule of reporter dye is liberated for each new molecule synthesized, and detection of the unquenched reporter dye provides the basis for quantitative interpretation of the data.

In real-time RT-PCR the amount of product formed, and the timing at which the product is formed, in the PCR reaction correlates with the amount of starting template. RT-PCR product will accumulate quicker in a sample having an increased level of mRNA compared to a standard or “normal” sample. Real-time RT-PCR measures either the fluorescence of DNA intercalating dyes such as Sybr Green into the synthesized PCR product, or can measure PCR product accumulation through a dual-labelled fluorigenic probe (i.e. TaqMan probe). The progression of the RT-PCR reaction can be monitored using PCR machines such as the Applied Biosystems' Prism 7000 or the Roche LightCycler which measure product accumulation in real-time. Real-time RT-PCR is compatible both with quantitative competitive PCR and with quantitative comparative PCR. The former uses an internal competitor for each target sequence for normalization, while the latter uses a normalization gene contained within the sample, or a housekeeping gene for RT-PCR.

The production and application of microarrays for measuring the level of expression of a gene at the transcriptional level are well known in the art. In general, in a microarray, a nucleotide sequence (for example an oligonucleotide, a cDNA, or genomic DNA) representing a portion, or all, of the Annexin A2 gene would occupy a known location on a substrate. Typically, the substrate includes a multitude of nucleotide sequences such that Annexin A2 and one or more other relevant genes can be assayed simultaneously. A nucleic acid target sample (for example total RNA or mRNA) obtained from a subject of interest is then hybridized to the microarray and the amount of target nucleic acid hybridized to each probe on the array is quantified and compared to the hybridisation which occurs to a standard or “normal” sample. One exemplary quantifying method is to use confocal microscope and fluorescent labels. The Affymetrix GeneChip™ Array system (Affymetrix, Santa Clara, Calif., USA) and the Atlas™ Human cDNA Expression Array system are particularly suitable for quantifying the hybridization; however, it will be apparent to those of skill in the art that any similar systems or other effectively equivalent detection methods can also be used. Fluorescently labelled cDNA probes may also represent the nucleic acid target sample. Such probes can be generated through incorporation of fluorescent nucleotides during reverse transcription of total RNA or mRNA extracted from a sample of the subject to be tested. Labelled cDNA probes applied to the microarray will hybridize with specificity to the equivalent spot of DNA on the array. Quantitation of hybridization of each arrayed element allows for assessment of corresponding mRNA abundance in the sample compared to the abundance observed in a standard or “normal” sample. With dual colour fluorescence, separately labelled cDNA probes generated from two sources of RNA are hybridized pairwise to the array. The relative abundance of the transcripts from the two sources corresponding to each specified gene is thus determined simultaneously. The miniaturized scale of the hybridization using microarray analysis affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels.

In the subject, the expression level of Annexin A2 may be measured directly in the subject, or in an alternative embodiment, the expression level of Annexin A2 may be measured in a sample obtained from a subject. It is to be made clear that the sample obtained from the subject that is analysed by the methods of the present invention may have previously been obtained from the subject, and, for example, may have been stored in an appropriate repository. In this instance, the sample would have been obtained from the subject in isolation of, and therefore separate to, the methods of the present invention. Accordingly, the methods of the aforementioned aspects of the invention can be practiced wholly in vitro.

The sample which is obtained from the subject may include, but is not limited to, a blood sample, or a sample derived from blood (for example a plasma or serum sample or a fraction of a blood, serum or plasma sample), cervical pap smears, ascites, bladder washing, uterine washing, and a tissue sample from one or both ovaries, one or both fallopian tubes, or metastatic tumour tissue of the subject. In certain circumstances, the sample may be manipulated in any way after procurement, such as by treatment with reagents, solubilization, or enrichment for certain components, such as the relevant protein or polynucleotide under investigation.

In one embodiment, the sample is a plasma sample obtained from blood of the subject. Blood plasma is the pale-yellow liquid component of blood, in which the blood cells in whole blood would normally be suspended. It makes up about 55% of the total blood volume. It is mostly water (up to 95% by volume) and contains dissolved proteins, glucose, clotting factors, mineral ions, hormones and carbon dioxide. Blood plasma is prepared by spinning a tube of fresh blood in a centrifuge until the blood cells fall to the bottom of the tube. The blood plasma is then poured or drawn off. Blood plasma, preferably supplemented with a clotting inhibitor, e.g. heparin or EDTA, has a density of approximately 1.025 kg/l. Blood serum is blood plasma without fibrinogen or the other clotting factors (i.e., whole blood minus both the cells and the clotting factors).

In the methods of the aforementioned aspects of the invention, once the expression level of Annexin A2 has been assessed in the subject, or in a sample obtained from the subject, the expression level is compared to a reference expression level for Annexin A2. The inventors have found that the expression level of Annexin A2 is higher in subjects with early stage ovarian cancer compared with normal subjects and compared with subjects with advanced ovarian cancer. Accordingly, an expression level of Annexin A2 in a subject which is higher than a reference expression level for Annexin A2 is indicative of early stage ovarian cancer in the subject, or indicates that the subject is susceptible to developing ovarian cancer. Reference herein to “higher” with respect to the expression level of Annexin A2, whether at the translational (protein) or transcriptional (mRNA) stage, is intended to mean, for example, at least about a 1%, 5%, 10%, 20%, 30%, 40%, 50%, 60%, 70%, 80%, 90%, 100%, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 5-fold, 10-fold, 20 fold, 50-fold, 100-fold increase in the expression level of Annexin A2 compared to the reference expression level.

In the context of the present invention, the reference expression level for Annexin A2 is a level of Annexin A2 which is known to be found in a subject not suffering from ovarian cancer (a “normal” subject in the context of the present invention). In this instance, a reference expression level for Annexin A2 may be derived from at least one normal subject and is preferably derived from an average of normal subjects (e.g. n=2 to 100 or more), wherein the subject or subjects have no prior history of ovarian cancer. A reference expression level for Annexin A2 can also be obtained from one or more normal samples from a subject suspected to have, or which has, ovarian cancer. For example, a reference expression level for Annexin A2 may be obtained from at least one normal sample and is preferably obtained from an average of normal samples (e.g. n=2 to 100 or more), wherein the subject is suspected of having, or which has, ovarian cancer. However, a reference expression level for Annexin A2 may also be a level that is associated with a particular advanced stage of ovarian cancer progression (i.e. stage III or IV). In this instance, the reference expression level for Annexin A2 would be derived from at least one subject having ovarian cancer at a defined advanced stage of progression, and would be preferably derived from an average of such subjects (e.g. n=2 to 100 or more).

In one embodiment, the reference expression level for Annexin A2 protein in plasma is about 276 ng/ml. Therefore, a subject with an expression level of Annexin A2 above this level indicates that the subject is highly likely to have early stage ovarian cancer or will be highly likely be susceptible to developing ovarian cancer.

In the methods of the aforementioned aspects of the invention, the expression level of one or more additional biomarkers known to be associated with ovarian cancer may also be assessed. Detecting the expression level of particular combinations of biomarkers may provide greater sensitivity, specificity and predictive power than any one biomarker alone. Accordingly, in some embodiments of the present invention, the expression level of Annexin A2 may be assessed in plasma of a subject in combination with the serum or plasma expression level of tumour associated antigen CA125 in the subject.

CA125 is encoded by the MUC16 gene and is a large transmembrane glycoprotein that was first identified using the monoclonal antibody OC125 in human ovarian carcinoma cell lines. The CA125 protein is characterised by a large extracellular domain containing up to 60 tandem repeats of 156 amino acids, and harbours the epitope for the original OC125 monoclonal antibody, as well as a heavily glycosylated amino terminal region. The molecular function of the CA125 protein has yet to be determined but it has been shown to function as a calcium dependent protease, and its presence in embryonic membranes as well as its heavily glycosylated extracellular domain may allow it to have a role in the motility and metastatic potential of ovarian carninoma.

The MUC16 gene has been found in a number of species, including human, rat, mouse, chimpanzee, cheetah, and monkey. MUC16 is also referred to in the art as CA125, mucin-16, CA125 ovarian cancer antigen, ovaran cancer-related tumour marker CA125 and ovarian carcinoma antigen CA125. Details regarding MUC16 may be accessed from the GenBank database at the National Center for Biotechnology Information (NCBI) (www.ncbi.nlm.nih.gov). For example, the Gene ID number for human MUC16 is 94025. The human MUC16 gene is represented by GenBank Accession Number NM_024690.2 and the encoded protein by NP_078966.2. Further details of the MUC16 gene in other species may be accessed from the NCBI. For example, the Gene ID number for rat MUC16 is 315451, for mouse is 73732, for chimpanzee is 744458, for cheetah is 106979012, and for monkey is 105705887.

Further details regarding the MUC16 gene in humans and other species can also be found at the UniGene portal of the NCBI (i.e. UniGene Hs. 432676—http://www.ncbi.nlm.nih.gov/UniGene/clust.cgi?ORG=Hs&CID=432676). Alternatively, details of the nucleotide and amino acid sequence for MUC16 can be accessed from the UniProt database (www.uniprot.org) wherein the UniProt ID for human MUC16 is Q8WXI7 (http://www.uniprot.org/uniprot/Q8WXI7).

As used herein, the term “subject” refers to any animal (e.g. a mammal), including, but not limited to humans, non-human primates, dogs, horses, cattle, sheep, pigs, rodents, and any other animal known to get ovarian cancer. Therefore, it should be appreciated that the methods of the present invention are not limited to humans. Details of Annexin A2 in other species, and their associated amino acid and mRNA sequences, may be readily accessed from the GenBank and UniProt databases (as discussed above) or sequences may be identified by BLAST searching using the human Annexin A2 sequence.

In a fourth aspect, the present invention provides a method of screening a candidate therapeutic agent for use in treating early stage ovarian cancer in a subject, the method including the step of assaying the candidate therapeutic agent for activity in reducing the expression level of Annexin A2 in plasma of the subject. As would be appreciated by a person skilled in the art, screening assays may be performed in vitro and/or in vivo.

In one embodiment, the method includes:

(a) administering the candidate therapeutic agent to the subject;

(b) assessing the expression level of Annexin A2 in plasma of the subject; and

(c) comparing the expression level of Annexin A2 in plasma of the subject with the expression level of Annexin A2 in plasma of an untreated subject having early stage ovarian cancer,

wherein if the expression level of Annexin A2 in the subject is lower than the expression level of Annexin A2 in the untreated subject, the candidate therapeutic agent is useful for treating early stage ovarian cancer.

In a further embodiment of the fourth aspect of the invention, the method includes:

(a) assessing the expression level of Annexin A2 in plasma of the subject who has been administered the candidate therapeutic agent; and

(b) comparing the expression level of Annexin A2 in plasma of the subject with the expression level of Annexin A2 in plasma of an untreated subject having early stage ovarian cancer,

wherein if the expression level of Annexin A2 in the subject is lower than the expression level of Annexin A2 in the untreated subject, the candidate therapeutic agent is useful for treating early stage ovarian cancer.

The expression level of Annexin A2 can be assessed in the subject using the methods described above. In some embodiments of the fourth aspect of the invention, the expression level of Annexin A2 is assessed in a plasma sample obtained from the subject.

In a further aspect, prospective agents may be screened to identify candidate therapeutic agents for the treatment of early stage ovarian cancer in a cell-based assay. In this regard, each prospective agent is incubated with cultured cells (for example cells obtained from an ovary of a normal subject or from a subject suffering from ovarian cancer, or cell lines derived from a normal or affected subject), and the expression level of Annexin A2 is assessed.

Accordingly, in a fifth aspect the present invention provides a method of screening a candidate therapeutic agent for use in treating early stage ovarian cancer in a subject, the method including:

(a) exposing the candidate therapeutic agent to a cell expressing Annexin A2;

(b) measuring for a change in the expression level of Annexin A2 in the cell; and

(c) comparing the expression level of Annexin A2 in the cell to a reference expression level for Annexin A2,

wherein if the expression level of Annexin A2 in the cell is lower than the reference expression level for Annexin A2, the candidate therapeutic agent is useful for treating early stage ovarian cancer in a subject.

Reference herein to “lower” with respect to the fourth and fifth aspects of the invention is intended to mean an expression level of Annexin A2, whether at the translational (protein) or transcriptional (mRNA) stage, that is at least about a 1%, 5%, 10%, 20%, 30%, 40%, 50%, 60%, 70%, 80%, 90%, 100%, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 5-fold, 10-fold, 20 fold, 50-fold, 100-fold decreased compared to the expression level in the untreated subject.

The reference expression level for Annexin A2 with respect to the fourth and fifth aspects of the invention may be determined as described above.

In another example, candidate therapeutic agents may be screened in organ culture-based assays. In this regard, each prospective agent is incubated with either a whole organ or a portion of an organ (such as a portion of an ovary of a normal or affected subject) derived from a non-human animal and modulation of the expression level of Annexin A2 measured.

The present invention also enables compositions which can be used to perform any one or more of the aforementioned methods of the invention. Accordingly, in a sixth aspect the present invention provides a composition for detecting early stage ovarian cancer in a subject, for identifying a subject having early stage ovarian cancer, or for determining if a subject is susceptible to developing early stage ovarian cancer, the composition including an agent that binds to, or interacts with, Annexin A2 present in plasma of the subject.

In one embodiment of the sixth aspect of the invention, the agent is an antibody specific for Annexin A2 protein. Details regarding Annexin A2 antibodies are provided above. In some embodiments, the composition further includes an agent that binds to, or interacts with, tumour associated antigen CA125.

In a seventh aspect, the present invention provides a kit for detecting early stage ovarian cancer in a subject, for identifying a subject having early stage ovarian cancer, or for determining if a subject is susceptible to developing early stage ovarian cancer, the kit including means for assessing the expression level of Annexin A2 in plasma of the subject.

In one embodiment of the seventh aspect of the invention, the kit includes an agent that binds to, or interacts with, Annexin A2 present in plasma of the subject. In some embodiments, the agent is an antibody specific for Annexin A2 protein. Details regarding Annexin A2 antibodies are provided above. In some embodiments, the kit further includes means for assessing the expression level of tumour associated antigen CA125 in serum or plasma of the subject.

In one embodiment of the seventh aspect of the invention, the kit includes a solid support, such as a chip, sensor, a microtiter plate or a bead or resin having a capture reagent attached thereon, wherein the capture reagent binds Annexin A2 protein and/or

CA125 protein. Therefore, for example, the kits of the present invention can comprise mass spectrometry probes for SELDI, such as ProteinChip® arrays, or a versatile fibre-based SPR sensing device. In the case of biospecfic capture reagents, the kit can comprise a solid support with a reactive surface, and a container comprising the biospecific capture reagent.

In some embodiments of the seventh aspect of the invention, the kit can also include a washing solution or instructions for making a washing solution, in which the combination of the capture reagent and the washing solution allows capture of Annexin A2 and/or CA125 protein on the solid support for subsequent detection by, for example, mass spectrometry. The kit may include more than one type of adsorbent, each present on a different solid support.

In some embodiments, such a kit can include instructions for suitable operational parameters in the form of a label or separate insert. For example, the instructions may inform a consumer about how to collect the sample, how to wash the probe or the particular protein or proteins to be detected.

In some embodiments, the kit can include one or more containers with samples that represent a reference expression level for each protein, and are therefore to be used as standards for calibration.

The term “about” as used in the specification means approximately or nearly and in the context of a numerical value or range set forth herein is meant to encompass variations of +/−10% or less, +/−5% or less, +/−1% or less, or +/−0.1% or less of and from the numerical value or range recited or claimed.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Finally, reference is made to standard textbooks of molecular biology that contain methods for carrying out basic techniques encompassed by the present invention. See, for example, Green M R and Sambrook J, 2012, supra.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

The invention is further illustrated in the following example. The example is for the purpose of describing particular embodiments only and is not intended to be limiting with respect to the above description.

### EXAMPLE 1

### Identification of Annexin A2 as a Marker for Ovarian Cancer

The inventors recently modelled the metastatic microenvironment of ovarian cancer in vitro and explored the two-way interactions between ovarian cancer and peritoneal cells using proteomics. This strategy identified several proteins that were specifically modulated by ovarian cancer cells when they interact with the peritoneum to maximise tumour cell attachment and survival. One of these proteins was the phospholipid calcium binding protein Annexin A2. Annexin A2 forms a complex with S100A10 and together they play a critical role in the plasminogen activator system. Annexin A2 binds with plasminogen and tissue plasminogen activator on the cell surface which leads to the conversion of plasminogen to plasmin, a key enzyme which facilitates essential cellular processes involved in cancer invasion and metastasis. The inventors have shown that Annexin A2 is highly expressed in 90% of serous ovarian cancers and is actively involved in the process of ovarian cancer metastasis. However, to date, no studies have investigated the potential of using Annexin A2 for the diagnosis of ovarian cancer. Furthermore, there has been no indication or expectation in the art that Annexin A2 may act as a plasma biomarker for early stage ovarian cancer.

### Methods

### Serum and Plasma Samples

Serum samples were collected from subjects with normal ovaries undergoing surgery for benign gynaecological conditions (n=30), subjects with benign ovarian tumours (n=37), early stage ovarian cancer subjects (stage I and II) (n=14) and advanced stage ovarian cancer subjects (stage III and IV) (n=72). Blood samples were collected into vacutainer plain tubes, the blood was allowed to clot, centrifuged at 3000 rpm for 10 minutes at room temperature and serum was stored at −80° C. until assayed.

Plasma samples were collected from subjects with normal ovaries undergoing surgery for benign gynaecological conditions (n=15), subjects with benign ovarian tumours (n=21), early stage ovarian cancer subjects (stage I and II) (n=11) and advanced stage ovarian cancer subjects (stage III and IV) (n=24). Blood samples were collected into vacutainer tubes containing the anticoagulant EDTA, the blood was centrifuged at 3000 rpm for 10 minutes at room temperature and plasma was stored at −80° C. until assayed.

### Annexin A2 ELISA Assay

Annexin A2 expression levels were measured using a commercial human Annexin A2 ELISA kit as per the manufacturer's instructions (USCN Life Science Inc., Wuhan, China). Briefly, 100 μl of standards and 10-fold dilutions of serum or plasma samples in PBS (pH 7.0) were added into each 96 well in duplicates. A capture Annexin A2 antibody was added and the absorbance reading measured using a plate reader at 450 nm. A standard curve of a different range of Annexin A2 antigen concentrations was performed in each plate and Annexin A2 concentration was determined from the standard curve. Expression of Annexin A2 was quantitatively measured in ng/ml. The detection limit of this assay was 0.321 ng/ml and the intra-assay and inter-assay coefficient was 6.1% and 3.3%, respectively.

### Results

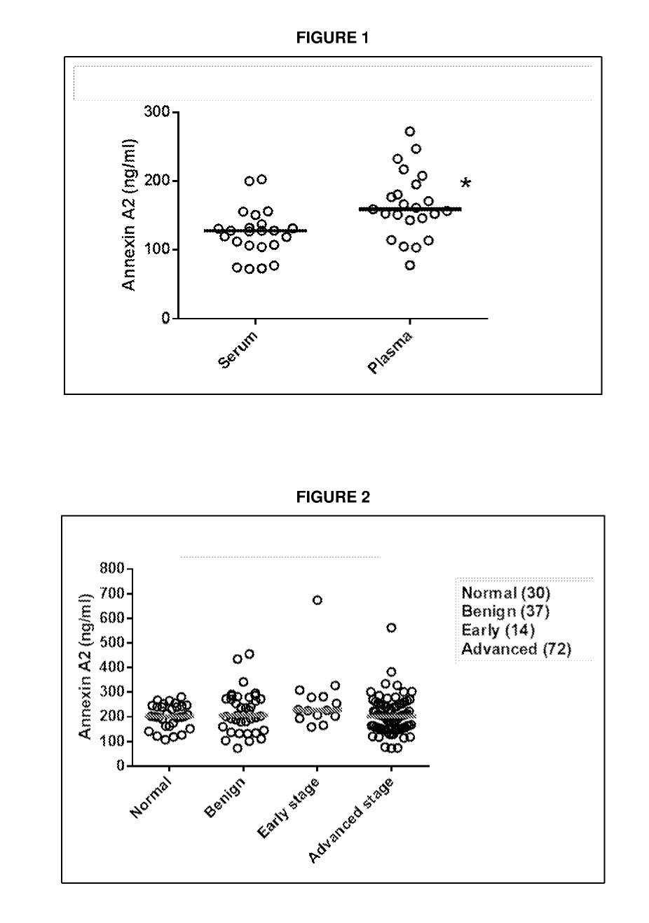

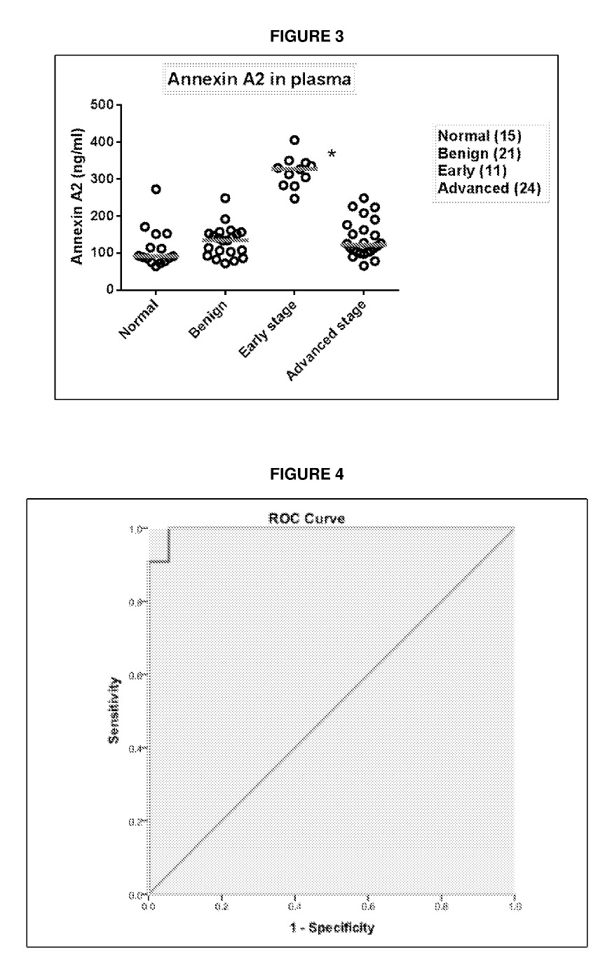

Annexin A2 expression levels in serum were 30% lower than in plasma (FIG. 1). Serum Annexin A2 expression levels did not differ amongst ovarian cancer and control subject groups (FIG. 2). However, surprisingly, plasma Annexin A2 expression levels were significantly elevated in early stage serous cancers compared to normal and benign groups (FIG. 3). Furthermore, plasma Annexin A2 expression levels were significantly elevated in early stage serous cancers compared advanced stage disease groups (FIG. 3). This represented a further surprising and unexpected result given that an elevated plasma Annexin A2 expression level in the early stages of ovarian cancer would imply that the elevated plasma Annexin A2 expression level would be maintained in advanced stages of the disease. However, this was not found to be the case. Ninety percent of subjects with early stage disease had plasma Annexin A2 expression levels greater than 300 ng/ml, whilst the non-cancer controls and the advanced stage group all had levels less than 300 ng/ml. A ROC curve of the non-cancer controls and early ovarian cancers was generated to determine the optimal cut-off value for plasma Annexin A2 (FIG. 4, AUC=0.995, P=0.001, CI 0.98-1.00, P<0.0001) and the best cut-off point, 276 ng/ml, had a sensitivity of 90.9% and a specificity of 100%. This is a marked improvement on current ovarian cancer markers such as CA125.

### Discussion

This is the first study showing that Annexin A2 can be used as a diagnostic marker for early stage ovarian cancer. Plasma expression levels of Annexin A2, but not serum expression levels, are increased in early stage ovarian cancer compared with healthy controls, subjects with benign ovarian tumours, and subjects with advanced stage disease. Using a cut-off point of 276 ng/ml, a sensitivity of 90.9% and a specificity of 100% was achieved for detecting early stage ovarian cancer.

Several studies have evaluated blood Annexin A2 levels in cancers of the liver, breast, and bowel, but results have been very inconsistent. Indeed, a recent study has found that serum Annexin A2 levels are reduced in patients with colon cancer. Most studies have measured Annexin A2 in serum and data on plasma Annexin A2 is very limited. Plasma is qualitatively different from serum in which fibrinogen is removed through clotting. During the clotting process many proteins including plasminogen are removed by adsorption to the fibrin clot. As Annexin A2 binds to plasminogen, its level is likely to be reduced by this process.

In conclusion, for the first time the surprising and unexpected utility of plasma Annexin A2 as a diagnostic marker in early stage ovarian cancer has been described.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

1. A method of detecting early stage ovarian cancer in a subject, the method including:
(a) assessing an expression level of Annexin A2 in plasma of the subject;
(b) comparing the expression level of Annexin A2 in the plasma with a reference expression level for Annexin A2; and
(c) detecting early stage ovarian cancer in the subject on the basis of the comparison, wherein an expression level of Annexin A2 in the subject that is higher than the reference expression level for Annexin A2 is indicative of early stage ovarian cancer in the subject.

2. The method according to claim 1, wherein the method is conducted in vitro.

3. The method according to claim 1, wherein the expression level of Annexin A2 is assessed in a plasma sample obtained from the subject.

4. The method according to claim 1, wherein the expression level of Annexin A2 protein is assessed.

5. The method according to claim 4, wherein the expression level of Annexin A2 protein is assessed using an antibody specific for the Annexin A2 protein.

6. The method according to claim 1, wherein the expression level of tumour associated antigen CA125 is also assessed in serum or plasma of the subject.

7. The method according to claim 6, wherein the expression level of CA125 is assessed in a serum or plasma sample obtained from the subject.

8. A method of identifying a subject having early stage ovarian cancer, the method including:
(a) assessing an expression level of Annexin A2 in plasma of the subject;
(b) comparing the expression level of Annexin A2 in the plasma with a reference expression level for Annexin A2; and
(c) identifying the subject as a subject having early stage ovarian cancer on the basis of the comparison,
wherein an expression level of Annexin A2 in the subject that is higher than the reference expression level for Annexin A2 indicatives that the subject has early stage ovarian cancer.

9. The method according to claim 8, wherein the method is conducted in vitro.

10. The method according to claim 8, wherein the expression level of Annexin A2 is assessed in a plasma sample obtained from the subject.

11. The method according to claim 8, wherein the expression level of Annexin A2 protein is assessed.

12. The method according to claim 11, wherein the expression level of Annexin A2 protein is assessed using an antibody specific for the Annexin A2 protein.

13. The method according to claim 8, wherein the expression level of tumour associated antigen CA125 is also assessed in serum or plasma of the subject.

14. The method according to claim 13, wherein the expression level of CA125 is assessed in a serum or plasma sample obtained from the subject.

15. A method of determining if a subject is susceptible to developing ovarian cancer, the method including:
(a) assessing an expression level of Annexin A2 in plasma of the subject;
(b) comparing the expression level of Annexin A2 in the plasma with a reference expression level for Annexin A2; and
(c) determining if the subject is susceptible to developing ovarian cancer on the basis of the comparison,
wherein an expression level of Annexin A2 in the subject that is higher than the reference expression level for Annexin A2 indicates that the subject is susceptible to developing ovarian cancer.

16. The method according to claim 15, wherein the method is conducted in vitro.

17. The method according to claim 15, wherein the expression level of Annexin A2 is assessed in a plasma sample obtained from the subject.

18. The method according to claim 15, wherein the expression level of Annexin A2 protein is assessed.

19. The method according to claim 18, wherein the expression level of Annexin A2 protein is assessed using an antibody specific for the Annexin A2 protein.

20-35. (canceled)
